(19) United States
(12) Patent Application Publication
(10) Pub. No.: US 2023/0381355 A1
(43) Pub. Date: Nov. 30, 2023

(54) METHODS AND APPARATUS FOR DISINFECTION OF FLUID-DISPENSING NOZZLES, ORIFICES AND THE LIKE

(71) Applicant: Acuva Technologies Inc., Burnaby (CA)

(72) Inventors: Babak Adeli KOUDEHI, Vancouver (CA); Majid KESHAVARZFATHY, Burnaby (CA); Milad Raeiszadeh OSKOUEI, Vancouver (CA)

(21) Appl. No.: 18/305,684

(22) Filed: Apr. 24, 2023

Related U.S. Application Data
(63) Continuation of application No. PCT/CA2021/051501, filed on Oct. 25, 2021.
(60) Provisional application No. 63/105,809, filed on Oct. 26, 2020.

(51) Int. Cl. A61L 2/10

(52) U.S. Cl. CPC A61L 2/10; A61L 2202/14; A61L 2202/121; A61L 2202/20

(57) ABSTRACT

An apparatus for disinfecting a surface of a fluid dispenser comprises a reflector and one or more UV radiation emitters. The reflector has a reflective surface extending around an exterior perimeter (e.g. circumferential) surface of the fluid dispenser. The UV radiation emitters are optically oriented to emit ultraviolet radiation toward the reflective surface. The reflector is configured to direct the ultraviolet radiation toward the exterior perimeter surface.

### REFERENCE TO RELATED APPLICATIONS

This application is a continuation of Patent Cooperation Treaty (PCT) application No. PCT/CA2021/051501 having an international filing date of 25 Oct. 2021, which in turn claims priority from, and for the purposes of the United States claims the benefit of 35 USC 119 in respect of, U.S. patent application No. 63/105,809 filed 26 Oct. 2021. All of the applications referred to in this paragraph are hereby incorporated herein by reference for all purposes.

### TECHNICAL FIELD

This invention relates generally to disinfection devices, and in particular to disinfection devices which include one or more ultraviolet (UV) radiation emitters configured to direct UV radiation toward the inner and/or outer surfaces of a tubular-shaped object (e.g. an object having a bore therethrough). Particular embodiments have example applications for disinfecting objects such as fluid-dispensing nozzles, orifices, spouts, faucets, and/or the like.

### BACKGROUND

UV radiation is known to be effective in neutralizing germs such as bacteria, viruses and fungi by damaging the DNA of the germs such that they become incapable of reproducing. Accordingly, it is common to use UV radiation for disinfection applications.

While it is known to use UV radiation for disinfection applications, state of the art UV disinfection devices are not designed to disinfect the dispensing parts of fluid-dispensing devices (e.g. tubes, nozzles, spouts, faucets, etc.). That is, some UV disinfection devices are not operable to disinfect the inner and/or outer surfaces of the dispensing part of fluid-dispensing devices.

The dispensing part of fluid-dispensing devices may be susceptible to contamination (e.g. secondary contamination or cross contamination between different users). Since fluid-dispensing devices are typically used frequently by multiple users, it is desirable to disinfect the dispensing part of fluid-dispensing devices frequently. Since the dispensing parts of fluid-dispensing devices are often tubular or hollow cylindrically shaped (e.g. they have a bore through them from which fluid is dispensed), it is desirable to direct UV radiation toward the exterior perimeter (e.g. exterior circumferential) surfaces of the dispensing parts of the fluid-dispensing devices. It may also be desirable to direct UV radiation toward other surfaces of such dispensing parts.

Some UV disinfection must be manually operated so it is not practical to disinfect fluid-dispensing devices using these manually operated UV disinfection devices.

There remains a need for UV disinfection devices that are effective for neutralizing germs on various surfaces of the dispensing parts of fluid-dispensing devices (e.g. nozzles, faucets, spouts, etc.). There remains a need for UV disinfection devices that are operable to disinfect an object (e.g. the dispensing parts of fluid-dispensing devices) from multiple angles at the same time. There also remains a need for cost-effective and/or energy efficient UV disinfection devices which possess such properties.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Aspects of the invention include without limitation:
disinfection devices comprising UV radiation emitting devices; and
UV disinfection devices configured to disinfect the fluid-dispensing parts of fluid-dispensing devices (e.g. nozzles, faucets, spouts, etc.).

One aspect of the invention provides an apparatus for disinfecting a surface of a fluid dispenser, the apparatus comprising: a reflector having a reflective surface extending around the fluid dispenser and configured to direct ultraviolet radiation toward the surface of the fluid dispenser; and one or more UV radiation emitters optically oriented to emit the ultraviolet radiation directed toward the reflective surface.

The fluid dispenser may be generally tubular shaped and the surface of the fluid dispenser may comprise an exterior perimeter (e.g. circumferential) surface of the fluid dispenser.

The one or more UV radiation emitters may comprise a plurality of UV-LEDs optically oriented to direct their respective principal optical axes toward the reflective surface.

The apparatus may further comprise a housing shaped to define a chamber extending around the exterior perimeter surface of the fluid dispenser, wherein the reflector and the plurality of UV-LEDs are located in the chamber.

The housing may comprise a slit extending around, and facing toward, the exterior perimeter surface of the fluid dispenser.

The apparatus may further comprise an insert made of a UV-transparent material, the insert received in the slit to close an interior of the chamber from the environment. The insert may be fabricated from quartz or fused silica.

The housing may comprise a generally annular shaped first portion and a second portion having the shape of a toroidal surface.

The reflector may be secured against the an interior surface of the second portion of the housing, and wherein the UV-LEDs may be mounted on a printed circuit board (PCB), the PCB is mounted on an interior surface of the first portion to direct the principal optical axes of the UV-LEDs toward the reflective surface of the reflector.

The interior surface of the second portion of the housing may be shaped to conform to the shape of the reflector.

The first portion and the second portion may be coupleable to one another.

The UV-LEDs may be evenly angularly spaced around the chamber.

The fluid dispenser may be generally tubular shaped and may have a central axis oriented in a direction of a bore of the tubular shape. The one or more UV radiation emitters comprise a UV-LED having a principal optical axis central to its direction of radiation emission. The principal optical axis of the UV-LED may be optically oriented toward the fluid dispenser and parallel to the central axis.

The UV-LED may be located or locatable at a location on the central axis.

The apparatus may comprise a stage operable to move the UV-LED away from the location when the fluid dispenser is in use.

The apparatus may further comprise a sleeve made of a UV transparent material attached to the exterior perimeter surface of the fluid dispenser. The sleeve may have an index of refraction in the range of 1.40 to 1.55.

The fluid dispenser may be a nozzle, a spout or a faucet of a fluid-dispensing device.

The reflective surface may comprise a concave surface extending around the exterior perimeter surface of the fluid dispenser.

The reflective surface may comprise a planar, elliptical or parabolic surface extending around the exterior perimeter surface of the fluid dispenser.

The apparatus may further comprise magnetic means, mechanical means, snap-together means and/or adhesive means for coupling the apparatus to the fluid dispenser or to another object so as to be suitably located adjacent to the fluid dispenser.

The apparatus may further comprise: a sensor for detecting a presence of a foreign object in the vicinity of the fluid dispenser; and control logic configured to turn the UV radiation emitters ON or OFF based on the output from the sensor.

The fluid dispenser may be generally tubular shaped and has a central axis oriented in a direction of a bore of the tubular shape; and the reflective surface may extend around an exterior perimeter surface of the fluid dispenser in a direction generally orthogonal to the central axis. The reflective surface may comprises a cross-sectional shape, in a cross-section orthogonal to the central axis, which is complementary to the external perimeter surface of the fluid dispenser. A cross-sectional shape, in a cross-section orthogonal to the central axis, of the external perimeter surface of the fluid dispenser may be circular. A cross-sectional shape, in a cross-section orthogonal to the central axis, of the external perimeter surface of the fluid dispenser may be non-circular (e.g. polygonal).

Another aspect of the invention provides a method for retrofitting the apparatus to a fluid-dispensing apparatus operable to dispense fluids from the fluid dispenser, the method comprising coupling the reflector to the fluid dispenser or to another object so as to be suitably located adjacent to the fluid dispenser.

Another aspect of the invention provides a fluid-dispensing apparatus comprising: a nozzle operative to dispense a fluid; a reflector having a reflective surface extending around an exterior perimeter (e.g. circumferential) surface of the nozzle and configured to direct ultraviolet radiation toward the exterior perimeter surface; and one or more UV radiation emitters optically oriented to emit the ultraviolet radiation toward the reflective surface.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a perspective view of a disinfection device according to an example embodiment of the invention. FIG. 1A is an exploded view of the disinfection device shown in FIG. 1. FIG. 1B is a partial cross-sectional perspective view of the disinfection device shown in FIG. 1.

FIG. 2 is a schematic side view showing the FIG. 1 disinfection device coupled to a nozzle to disinfect the nozzle.

FIG. 3 depicts some characteristics of an example radiation pattern emitted by the UV emitters of the disinfection device shown in FIG. 1.

FIG. 4 is a partial perspective view of a disinfection system according to an example embodiment of the invention. FIG. 4A is a perspective view of a UV reflector of the disinfection system shown in FIG. 4. FIG. 4B is a side cross-sectional view of the UV reflector of the disinfection system shown in FIG. 4.

FIG. 5 is a schematic side cross-sectional view of a disinfection system comprising a quartz sleeve according to an example embodiment of the invention.

FIGS. 6A-B are partial perspective and cross-sectional views of exemplary stages which may be provided as part of the FIG. 4 or FIG. 5 disinfection systems.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

One aspect of the invention provides systems and methods for disinfecting one or more surfaces of a fluid-dispensing part (e.g. a nozzle, faucet, spout and/or the like) of a fluid-dispensing device. Such surfaces may comprise, by way of non-limiting example, an exterior perimeter (e.g. circumferential) surface, a bore-defining surface and/or end surface(s) of the fluid-dispensing part. Another aspect of the invention provides systems and methods for disinfecting three-dimensional objects from multiple angles and/or one or more continuous ranges of angles. Systems and devices described herein may be operated to automatically disinfect the exterior perimeter (e.g. circumferential) surface, the bore-defining surface and/or the end surface(s) of fluid dispensers when they are not in use. Systems and devices described herein comprise one or more ultraviolet (UV) radiation emitters operable to emit UV radiation. The UV radiation emitters may be optically oriented to face toward a UV reflector extending around the exterior perimeter surface of the object.

Unless context dictates otherwise, the term “optically oriented” (as used herein) should be interpreted to imply that UV radiation emitters described herein may include any number (e.g. 0, 1, 2, 3, etc.) of suitable optical elements (e.g. lenses, waveguides, etc.) located in the optical path between a UV radiation source and an output location of the UV radiation emitter. That is, a UV radiation emitter optically oriented to direct UV radiation toward an object should be understood to include a UV radiation emitter which comprises any number of suitable optical elements located in the optical path between a UV radiation source and an output location of the UV radiation emitter. Unless context dictates otherwise, the term UV radiation emitter (as used herein) should be understood to refer to devices which emit electromagnetic radiation that comprises at least some radiation having a wavelength shorter than that of the visible spectrum. Unless context dictates otherwise, the term UV reflector (as used herein) should be understood to refer to objects that have a surface that is either specularly reflective (e.g. reflectivity over 50% in some embodiments; and over 60% in some embodiments) or diffusely reflective (e.g. reflectivity over 50% in some embodiments; and over 60% in some embodiments) to electromagnetic radiation having a wavelength shorter than that of the visible spectrum. For brevity, this disclosure and/or the accompanying claims may refer to the shape of a UV reflector. Unless context dictates otherwise, the shape of a UV reflector should be understood to refer to the shape of the reflective surface of the reflector.

UV reflectors described herein may have the shape of a toroidal surface. Unless context dictates otherwise, the term “toroidal surface” (as used herein) means a surface of revolution with a hole in the middle. A toroidal surface can be generated, for example, by sweeping a closed or open curve around an axis of revolution that is in a plane of the curve and spaced from the boundary of the curve (i.e. the axis of revolution does not intersect the curve). The curve may be a quarter-circle, a quarter-ellipse, a half-parabola, or any freeform curve.

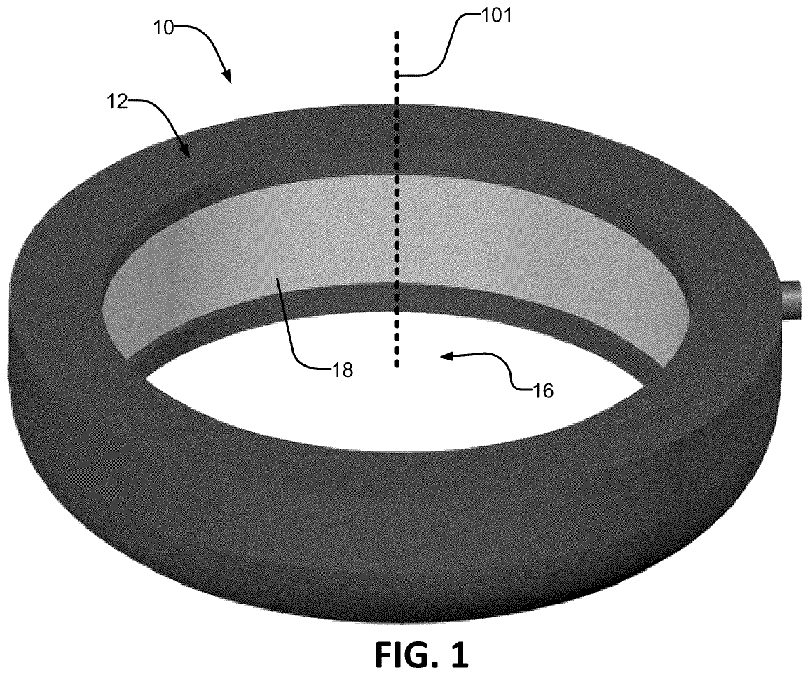

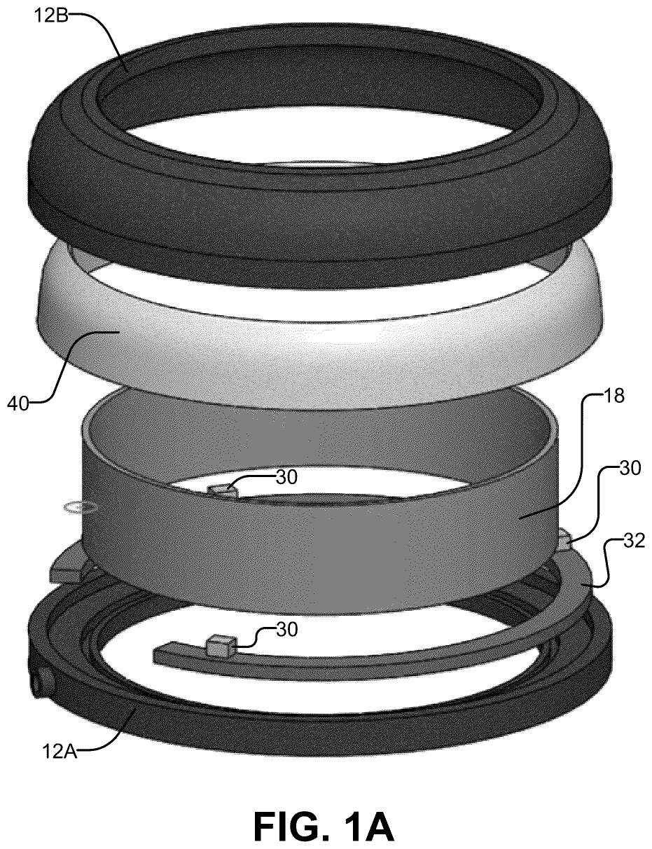

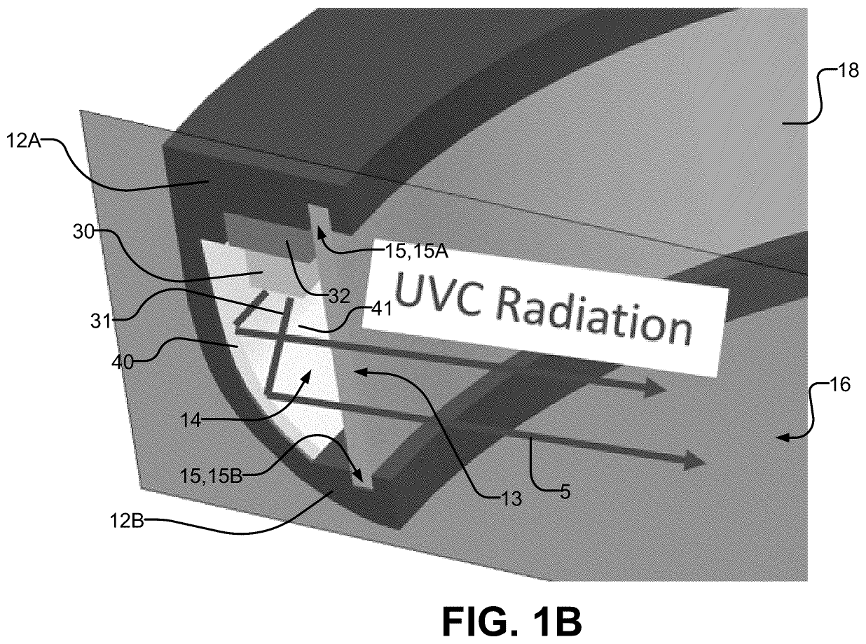

Aspects of the invention provide UV reflectors which are shaped to define a generally cylindrically-shaped disinfection region (e.g. see FIG. 1) which is encircled by the UV reflector. Such UV reflectors are shaped to direct UV radiation emitted by one or more UV radiation emitters toward the disinfection region. Such UV reflectors may be shaped and/or located to direct UV radiation toward various surface(s) of a fluid-dispensing part located in a disinfection region. Such surfaces of the fluid-dispensing part, may include, for example, the exterior perimeter (e.g. circumferential) surface, the bore-defining surface and/or one or more end surface(s) of the fluid-dispensing part. Further aspects of the invention are described herein with reference to various exemplary embodiments of the invention described below.

FIG. 1 is a perspective view of an ultraviolet (UV) disinfection device 10 according to an example embodiment of the invention. FIG. 1A is an exploded view and FIG. 1B is a partial cross-sectional perspective view showing various components of UV disinfection device 10. UV disinfection device 10 comprises one or more UV radiation emitters 30 and a UV reflector 40 located inside of a housing 12. Housing 12 is shaped to define a chamber 14 (which houses UV radiation emitters 30 and UV reflector 40) extending (e.g. circumferentially) around a generally cylindrically-shaped disinfection region 16 (e.g. see FIG. 1B). An object 2 such as a fluid dispenser having an exterior perimeter (e.g. circumferential) surface 2A may be positioned in disinfection region 16. UV radiation emitters 30 and UV reflector 40 are arranged within chamber 14 (see FIG. 1B) to direct UV radiation 5, either via direct emission or via reflection, toward disinfection region 16 to disinfect various surface(s) of object 2. UV reflector 40 has a reflective surface 41 extending around disinfection region 16 and located and/or shaped to direct ultraviolet radiation 5 received from UV radiation emitters 30 toward exterior perimeter surface 2A.

Housing 12 may be shaped to define a toroidal-shaped (e.g. ring-shaped, annular-shaped, etc.) chamber 14 encircling disinfection region 16. Reflector 40 may have the general shape of a toroidal surface as described in more detail elsewhere herein.

Housing 12 may be at least partially made of a UV-transparent material or may otherwise comprise one or more UV-transparent (e.g. quartz) surfaces or slits 13 (i.e. openings) facing toward disinfection region 16. For example, housing 12 may comprise a UV transparent window (facing toward disinfection region 16) which may be integrally formed with or coupled to the non-UV transparent surfaces of housing 12.

In the example embodiment shown in FIG. 1B, housing 12 comprises a slit 13 facing toward disinfection region 16. Slit 13 may extend circumferentially around a central axis 101 (e.g. an axis of symmetry) of UV disinfection device 10. Central axis 101 typically passes through disinfection region 16. Advantageously housing 12 may be made with low cost materials such as polycarbonate, ABS plastic, or the like where housing 12 comprises slit 13. That is, housing 12 does not need to be made of or comprise a UV transparent material as slit 13 provides an optical window for UV disinfection device 10 to direct UV radiation 5 toward disinfection region 16.

In embodiments where housing 12 comprises a slit 13, housing 12 may comprise tracks 15. Like slit 13, tracks 15 may extend circumferentially around central axis 101. Tracks 15 may be provided on an upper portion 12A of housing 12, a lower portion 12B of housing 12, or both. Tracks 15 may comprise a circumferentially extending indentation, channel, groove and/or the like (e.g. see FIG. 1B) shaped to receive an insert 18 made of suitable UV transparent materials such as fused silica, quartz, etc. Like slit 13 and tracks insert 18 may extend circumferentially around central axis 101. Insert 18 may be secured between upper and lower tracks 15A, 15B to cover slit 13 (e.g. see FIG. 1B). Additionally or alternatively, insert 18 may be adhered (e.g. with an adhesive) between upper portion 12A and lower portion 12B to cover slit 13. Insert 18 may cover slit 13 to enclose chamber 14 from the environment. Enclosing chamber 14 from the environment can advantageously protect some of the internal components of UV disinfection device 10 (e.g. UV emitters 30, reflector 40, etc.) from exposure to moisture or unwanted debris in the environment.

Insert 18 may be shaped or otherwise configured to help UV disinfection device 10 deliver a desirable radiation pattern to disinfection region 16. For example, insert 18 may be or may comprise a planar lens, a concave lens, a convex lens, a plano-convex lens, a plano-concave lens, a biconvex lens, a biconcave lens, etc.

Advantageously UV disinfection device 10, due to its shape and/or its location, may be operable to direct UV radiation 5 toward disinfection region 16 from a wide range of angles (e.g. angles up to 360°) and/or a continuous range of angles. That is, UV disinfection device 10 may be operated to disinfect object 2 located in disinfection region 16 from multiple angles simultaneously.

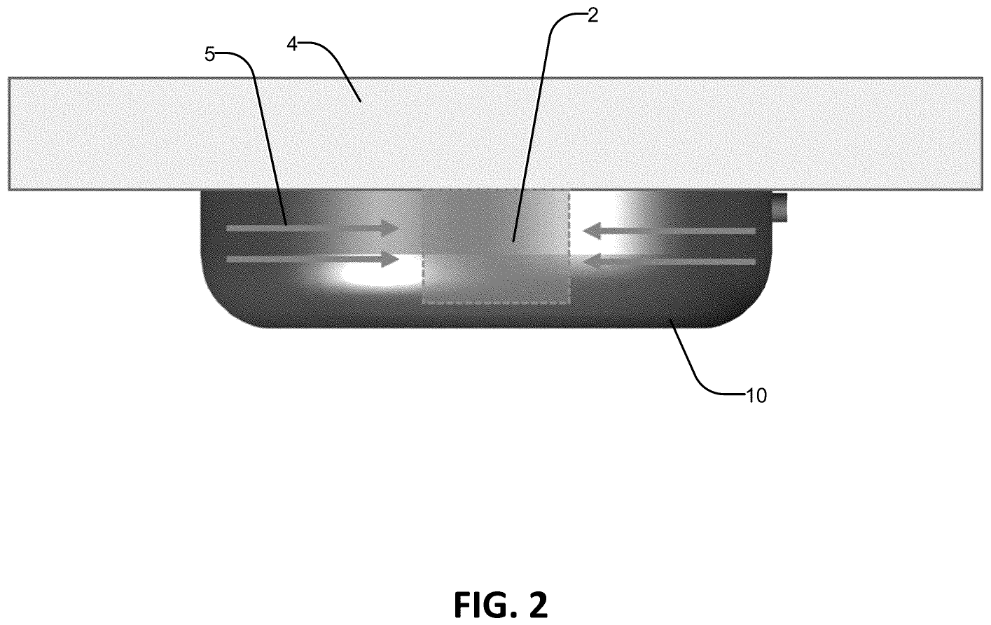

UV disinfection device 10 has example applications for disinfecting various surface(s) of objects 2 (e.g. fluid-dispensing parts 2, such as nozzles, fluid-dispensing orifices, and/or the like) placed or otherwise located in disinfection region 16 (e.g. see FIG. 2). For example, UV disinfection device 10 may be coupled to (e.g. disposed around) a fluid-dispensing nozzle 2 to disinfect nozzle 2 when it is not in use. For these example applications, UV disinfection device 10 has an internal radius which is typically less than about 3 cm, an external radius which is typically less than about 5 cm, and a height which is typically less than about 10 cm. The dimensions of UV disinfection device 10 may be modified to any suitable size to provide a sufficiently large disinfection region 16 for a specific application.

As described above, UV disinfection device 10 comprises a plurality of UV radiation emitters 30 which are housed inside of housing 12. UV radiation emitters 30 may be operated to emit UV radiation 5, either directly or via reflection, toward disinfection region 16 to disinfect an object 2 located in disinfection region 16. UV radiation emitters 30 may be operated to emit UV radiation 5 having wavelengths which are in the UV-C range (i.e. wavelengths that are particularly effective for neutralizing germs such as viruses, bacteria, etc.) and/or in the UV-A range (e.g. when object 2 is coated with a photocatalyst). In some embodiments or applications, UV radiation emitters 30 may be designed to emit UV radiation 5 having wavelengths which are on the order of about 100 nm to about 500 nm. In some embodiments, this UV radiation comprises UV-C radiation (˜200-300 nm) and/or UV-A radiation (˜300-400 nm)

UV radiation emitters 30 may comprise any suitable type of UV radiation emitting devices. In a currently preferred embodiment, UV radiation emitters 30 comprise UV light emitting diodes (LEDs) (i.e. solid state radiation sources which release photons when an electric potential is applied across the radiation source). Advantageously, UV-LEDs may be smaller, more energy efficient, less expensive to manufacture and/or more environmentally friendly than other types of UV emitting devices.

UV radiation emitters 30 may be mounted on a printed circuit board (PCB) 32 as shown in FIG. 1B. PCB 32 may comprise electronic controllers (e.g. microcontrollers) and suitable driving circuitry operable to control UV emitters 30. The electronic controllers of PCB 32 may be configured to control UV emitters 30 in a variety of different ways. For example, the electronic controllers of PCB 32 may be configured to control the intensity of some or all of UV radiation emitters 30. As another example, the electronic controllers of PCB 32 may be configured to control the number of UV radiation emitters 30 which are turned ON or OFF at the same time to reduce the amount of energy consumed by UV disinfection device 10.

In some embodiments, UV disinfection device 10 comprises a suitable sensor or detector configured to detect motion and/or the presence of foreign objects in or near to disinfection area 16 and the electronic controllers of PCB 32 are configured to turn OFF certain UV radiation emitters 30 upon detection of such motion or foreign object in or near to disinfection region 16. For example, the electronic controllers of PCB 32 may be configured to turn OFF certain UV radiation emitters 30 when the detector detects a foreign object approaching disinfection region 16 (e.g. a cup being placed in the vicinity of nozzle 2).

PCB 32 may be configured to receive an external control signal (e.g. a wireless control signal) from an external controller such as software installed on a computer, a mobile application installed on a smartphone, etc. PCB 32 may control UV radiation emitters 30 based on the external control signal.

As shown in FIGS. 1A and 1B, UV radiation emitters 30 may be spread around chamber 14 and operated to emit UV radiation 5 from various locations (relative to object 2 provided in disinfection region 16). That is, UV radiation emitters 30 may be spread around chamber 14 and operated to direct UV radiation 5 toward various surface(s) (e.g. the exterior perimeter surface 2A) of object 2 in disinfection region 16 from a variety of different angles relative to object 2.

In some embodiments, UV radiation emitters 30 are evenly angularly spaced around chamber 14 (e.g. about axis 101). For example, UV disinfection device 10 may comprise four UV radiation emitters 30 which are spaced at about 90 degrees apart from each other about central axis 101. As another example UV disinfection device 10 may comprise six UV radiation emitters 30 which are spaced at about 60 degrees apart from each other about central axis 101. UV radiation emitters 30 may be spaced angularly evenly around chamber 14 to help create a cylindrically symmetric radiation pattern around central axis 101.

In other embodiments, UV radiation emitters 30 are spaced in an uneven manner around chamber 14 (i.e. chamber 14 comprises regions which have relatively more UV emitters 30 and/or regions which have relatively fewer UV radiation emitters 30). For example, UV disinfection device 10 may comprise a first set of UV radiation emitters 30 spaced apart from a second set of UV radiation emitters 30 (e.g. by about 180 degrees apart from each other about central axis 101). The first set may comprise a greater number of UV radiation emitters 30 than the second set. The first set may collectively emit greater amounts of UV radiation 5 compared to the second set. In such embodiments, UV disinfection device 10 may be located (e.g. pivoted around central axis 101 and relative to object 2) to position the first set of UV emitters 30 at a location which is more proximate to sub-regions in disinfection region 16 for which greater amounts of UV radiation are desired.

Preferably UV disinfection device 10 comprises one or more UV reflectors 40 disposed inside of housing 12. UV reflector 40 may comprise a dominantly specular reflective surface 41 made of or otherwise coated with materials suitable for reflecting UV radiation 5 emitted by UV radiation emitters 30 in a specular fashion. Examples of suitable materials include, but are not limited to aluminum, PTFE, etc. Additionally or alternatively, UV reflector 40 may comprise a dominantly diffuse reflective surface 41 made of or otherwise coated with materials suitable for reflecting UV radiation 5 emitted by UV radiation emitters 30 in a diffuse fashion. Examples of suitable materials include, but are not limited to aluminum, Polytetrafluoroethylene (PTFE), polycrystalline materials, etc.

UV reflectors 40 are located adjacent to UV radiation emitters 30 and oriented to help direct UV radiation 5 emitted by UV radiation emitters 30 toward disinfection region 16. In the example embodiment shown in FIG. 1A, UV disinfection device 10 comprises a UV reflector 40 having the shape of a toroidal surface. That is, UV reflector 40 has a three-dimensional shape defined by sweeping a smooth and continuous arc around central axis 101 (i.e. an axis of revolution that is in a plane of the arc and spaced from the boundary of the arc).

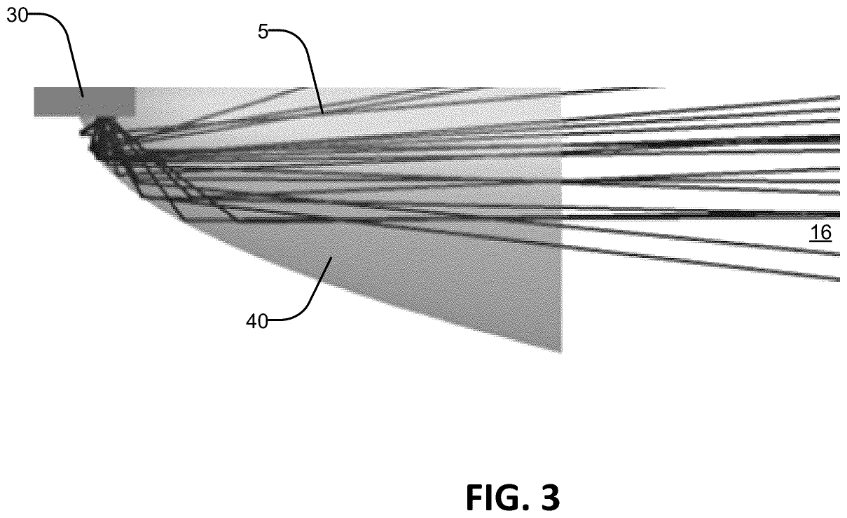

FIG. 3 is a schematic side cross-sectional view depicting some characteristics of an example radiation pattern emitted by a UV disinfection device 10 comprising a UV reflector having a concave reflective surface 2A extending around (e.g. circumferentially around) object 2 and disinfection region 16. In the example embodiment shown in FIG. 3, UV reflector 40 has the cross-sectional shape of a quarter-ellipse (i.e. the arc which is swept around central axis 101 to define the shape of UV reflector 40 has the shape of a quarter-ellipse). UV reflector 40 may take on other cross-sectional shapes (e.g. a quarter-circle, a line, a half-parabola, or any other suitable smooth and continuous function) in other embodiments.

As depicted in FIG. 3, UV reflector 40 may be suitably shaped and suitably oriented relative to UV radiation emitters 30 to help concentrate or otherwise direct UV radiation 5 emitted from UV emitters 30 toward disinfection region 16. For example, UV radiation emitters 30 may in some cases be located adjacent to UV reflector 40 and oriented such that their principal optical axis 31 is directed toward reflective surface 41 of UV reflector 40 (e.g. see FIG. 1B) instead of towards disinfection region 16. In these cases, UV radiation 5 emitted by UV radiation emitters 30 along principal optical axis 31 may impinge directly on reflective surface 41 or may be optically oriented to impinge on reflective surface 30 by one or more suitable optical elements (not shown).

In some embodiments, housing 12 comprises a first portion 12A and a second portion 12B (see FIG. 1A, for example). In these embodiments, first portion 12A and second portion 12B may be coupleable to each other. Fabricating housing 12 from multiple coupleable components (e.g. first and second housing portions 12A, 12B) may provide advantages such as ease of manufacture, assembly and/or storage.

First portion 12A may have the general shape of an annulus (i.e. a generally flattened disk with a hole in the middle). First portion 12A may comprise means for coupling UV disinfection device 10 to object 2 or to another object (e.g. a base 4 from which object 4 extends—see FIG. 2) so that UV disinfection device 10 is suitably located adjacent to object 2. For example, first portion 12A may comprise magnets which can releasably hold a surface of first portion 12A against a metallic base 4 from which object 2 extends. As another example, first portion 12A may comprise or otherwise receive suitable adhesives which can adhere first portion 12A to base 4 from which object 2 extends. As still another example, first portion 12A may form a “snap-together” fit with object 2 or with another object so that UV disinfection device 10 is suitably located adjacent to object 2. Such a snap-together fitting with object 2 may involve forcing disinfection device 10 against object 2, so that first portion 12A deforms under such force and, after such deformation, exerts restorative force which tends to hold UV disinfection device 10 against object 2, thereby coupling disinfection device 10 to object 2. As still another example, first portion 12A may comprise a suitable mechanism for coupling to object 2 or to another object so that UV disinfection device 10 is suitably located adjacent to object 2. UV radiation emitters 30 and/or PCB 32 may be mounted to a surface of upper portion 12A as shown in FIG. 1B.

Second portion 12B may have the shape of a toroidal surface (as defined above). That is, second portion 12B may have the shape of a shell generated by sweeping a curve or an arc around central axis 101. Second portion 12B may be shaped to conform to the shape of reflector 40. That is, second portion 12B may be shaped to allow UV reflector 40 to fit snuggly against an interior surface of second portion 12B.

Although not necessary, UV disinfection device 10 may be embodied as a standalone device which may be detachably coupled to object 2 or to another object (e.g. base 4) so that disinfection device 10 is suitably located in a vicinity of object 2. In some such embodiments UV disinfection device 10 may be attached to a base 4 from which object 2 extends so that object 2 is located in disinfection region 16.

Alternatively, UV disinfection device 10 may be embodied as part of object 2 or as part of another object (e.g. base 4 from which object 2 extends) so that UV disinfection device 10 is suitably located in a vicinity of object 2. In some such embodiments UV disinfection device 10 may be integrally formed with base 4 from which object 2 extends so that disinfection device 10 is suitably located in a vicinity of object 2. For example, UV disinfection device 10 may be embodied as part of a fluid-dispensing device comprising a fluid-dispensing nozzle 2.

UV disinfection device 10 may in some cases be embodied as a kit. That is, UV disinfection device 10 may be embodied as a collection of components that can be delivered to and/or assembled at the location of object 2 or any other suitable location to form all or parts of UV disinfection device 10. Such a kit typically comprises one or a combination of components which form the UV disinfection device 10 described herein. Examples of such components include but are not limited to: housing 12 (e.g. first housing portion 12A and second housing portion 12B), insert 18, UV emitter 30, PCB 32, and UV reflector 40.

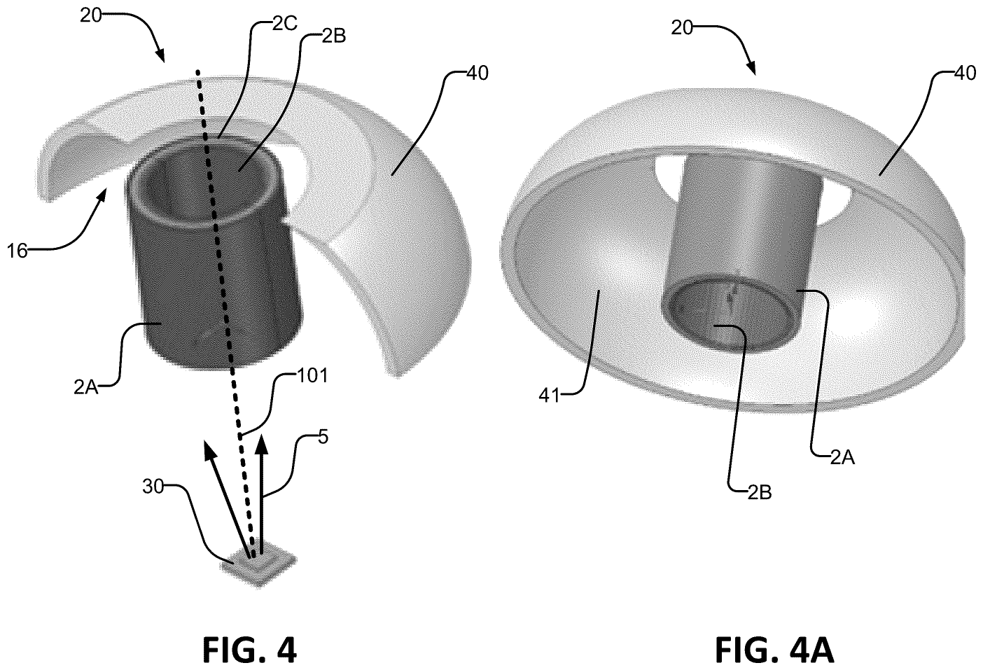

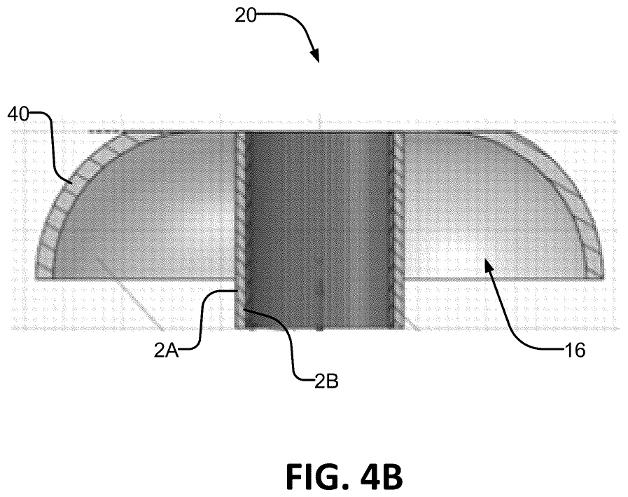

FIG. 4 is a perspective view of a UV disinfection system 20 according to an example embodiment of the invention. Like UV disinfection device 10, UV disinfection system 20 comprises a UV radiation emitter 30 and a UV reflector 40 having a reflective surface 41 extending around object 2 (e.g. around an exterior perimeter (e.g. circumferential) surface 2A of object 2). UV reflector 40 may be disposed around object 2 to direct UV radiation 5 emitted by UV radiation emitter 30 toward various surface(s) of object 2 located in disinfection region 16. Such surfaces of object 2, may include, for example, the exterior perimeter surface 2A, the bore-defining surface 2B and/or one or more end surface(s) 2C of object 2. UV radiation emitter 30 may be provided at a location which is relatively distal from UV reflector 40 and/or disinfection region 16 (i.e. in comparison to UV disinfection device 10 which has UV emitters 30 located adjacent to reflector 40). UV radiation emitter 30 may be located along central axis 101 as shown in FIG. 4. UV radiation emitter 30 may be optically oriented to emit UV radiation 5 directly toward object 2. That is UV radiation emitter 30 may be optically oriented so that its principal optical axis faces toward object 2.

Advantageously UV disinfection system 20 may be operated in some cases to disinfect exterior perimeter (e.g. circumferential) surface 2A, bore-defining surface 2B and one or more end surfaces 2C of a tubular shaped object 2 (e.g. a nozzle, a fluid-dispensing orifice, and/or the like having a bore through which fluid is dispensed) at the same time. As described above, UV radiation emitter 30 may be located along the central axis 101 of a tubular shaped object 2 and oriented to direct UV radiation 5 directly toward bore-defining surface 2B and one of the end surfaces 2C of object 2. In the FIG. 4 embodiment, UV reflector 40 is disposed around the exterior of object 2 and shaped to reflect UV radiation 5 emitted by UV radiation emitter 30 toward the exterior perimeter (e.g. circumferential) surface 2A and other end surface 2C of object 2. Note that the exterior perimeter surface 2A of object 2 need not be cylindrical and may generally have any suitable perimeter shape.

In some embodiments, the exterior perimeter surface 2A, bore-defining surface 2B and/or end surface(s) 2C of object 2 are coated with or otherwise comprise a layer of UV reflective material such as aluminium, Teflon, or the like, to enhance the disinfection effect. In embodiments where exterior perimeter surface 2A is UV reflective, UV radiation 5 which has been reflected towards exterior surface 2A by the UV reflector 40 may be reflected back towards reflector 40 by the reflective surface of exterior perimeter surface 2A, and then back again towards the exterior perimeter surface 2A from UV reflector 40 (i.e. UV radiation may be reflected back and forth between UV reflector 40 and exterior surface 2A). Since UV energy is not substantially attenuated as UV radiation 5 reflects back and forth between UV reflector 40 and exterior surface 2A, the multiple reflections between the UV reflector 40 and the exterior perimeter surface 2A can in some cases further intensify the disinfection process.

Similarly, in embodiments where interior surface 2B is UV reflective, UV radiation 5 (emitted by UV radiation emitter 30) that impinges on interior surface 2B may undergo several reflections between different parts of interior surface 2B to further intensify the disinfection process.

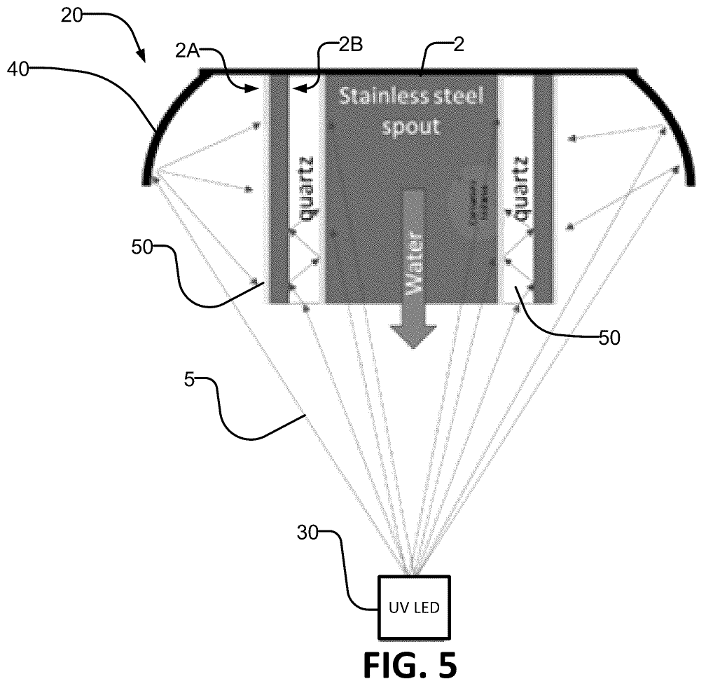

In some embodiments, exterior surface 2A, bore-defining surface 2B and/or end surface(s) 2C of object 2 are coated with a UV transparent material. In some embodiments, system 20 comprises a sleeve 50 attached to or otherwise provided at the exterior surface 2A and/or interior surface 2B of object 2 (e.g. see FIG. 5). Sleeve 50 may be fabricated from of a UV transparent material, such as quartz and/or the like. Sleeve 50 may have an index of refraction which is selected to cause UV radiation 5 traveling in sleeve 50 to undergo total internal reflection (TIR) at the interface between sleeve 50 and air. For example, sleeve 50 may have an index of refraction which is in the range of 1.4 to 1.55. If contaminants (e.g., biofilm, stain, etc.) form on the surface of sleeve 50, the refractive index at the interface of sleeve 50 and ambient air changes (i.e. due to the presence of the contaminants) to impair the TIR phenomenon and thereby cause UV rays 5 in sleeve 50 to exit sleeve 50. UV rays 5 which exit sleeve 50 can disinfect the contaminated areas on the surface of sleeve 50.

In some embodiments, exterior surface 2A, bore-defining surface 2B and/or end surface(s) of object 2 are coated with or otherwise comprises a thin film of a photosensitizer, such as a photocatalyst, to enhance the disinfection effect. If the exterior surface 2A and/or interior surface 2B of object 2 comprises a sleeve 50, the photosensitizer may be coated on the surface of sleeve 50. Where object 2 is coated with or otherwise comprises a photosensitizer, photo-initiated oxidative species may be formed on the photocatalytic surface to create a self-disinfecting surface. That is, the photosensitizer (e.g. photocatalysts such as titanium dioxide) may generate radicals (i.e. chemical species that are highly active and react fast with other molecules), when activated by UV radiation in the presence of oxygen or water, which react with contaminants to eliminate the contaminants. Where object 2 is coated with or otherwise comprises a photosensitizer, UV radiation emitter 30 may comprise UVA-LEDs (i.e. LEDS with emit radiation having wavelengths in the range of 315 nm to 400 nm).

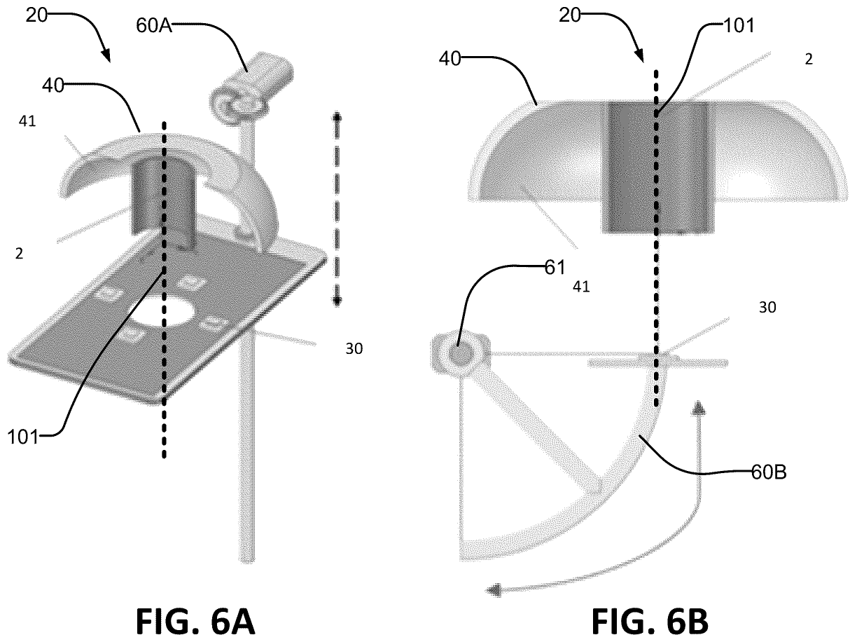

System 20 may include mechanisms for moving the UV radiation emitter 30 to a desirable position (e.g. a position along central axis 101 of object 2 and spaced apart from object 2) to emit UV radiation 5 toward object 2 and/or reflector 40. For example, system 20 may include movable stages 60 which may be operated to adjust the position of UV radiation emitter 30 relative to the position of object 2 and/or reflector 40. In the example embodiment shown in FIG. 6A, UV radiation emitters 30 are mounted on a stage 60A which may be operated to move UV radiation emitters 30 toward or away from object 2 in directions which are parallel to central axis 101. In the example embodiment shown in FIG. 6B, UV radiation emitters 30 are mounted on a stage 60B which may be operated to move UV radiation emitters 30 toward or away from object 2 by pivoting stage 60B relative to a base 61 about an axis which may be orthogonal to central axis 101.

In operation, stage 60 holding UV radiation emitters 30 may be moved toward fluid dispenser 2 when it is desirable to disinfect fluid dispenser 2 and away from fluid dispenser 2 when it is desirable to provide space near the vicinity of fluid dispenser 2. For example, stage 60 may be operated to move UV radiation emitters 30 away from fluid dispenser 2 whenever fluid dispenser 2 is operated (e.g. by a person) to dispense liquids, and toward fluid dispenser 2 after the liquid has been dispensed to disinfect fluid dispenser 2 immediately thereafter. This way stage 60 and UV radiation emitters 30 do not obstruct the operation of fluid dispenser 2 when it is in use.

Controllers (e.g. controllers of PCB 32) described herein may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, and the like. For example, one or more data processors in a control circuit for a device may implement methods as described (e.g. automatically controlling valves with a controller) herein by executing software instructions in a program memory accessible to the processors. It may be convenient to use a commercially available PLC for controllers described herein.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. An apparatus for disinfecting a surface of a fluid dispenser, the apparatus comprising:
a reflector having a reflective surface extending around the fluid dispenser and configured to direct ultraviolet radiation toward the surface of the fluid dispenser; and
one or more UV radiation emitters optically oriented to emit the ultraviolet radiation directed toward the reflective surface.

2. An apparatus according to claim 1 herein the fluid dispenser is generally tubular shaped and the surface of the fluid dispenser comprises an exterior perimeter (e.g. circumferential) surface of the fluid dispenser.

3. The apparatus of claim 2 wherein the one or more UV radiation emitters comprise a plurality of UV-LEDs optically oriented to direct their respective principal optical axes toward the reflective surface.

4. The apparatus of claim 3 further comprising a housing shaped to define a chamber extending around the exterior perimeter surface of the fluid dispenser, wherein the reflector and the plurality of UV-LEDs are located in the chamber.

5. The apparatus of claim 4 wherein the housing comprises a slit extending around, and facing toward, the exterior perimeter surface of the fluid dispenser.

6. The apparatus of claim 5 further comprising an insert made of a UV-transparent material, the insert received in the slit to close an interior of the chamber from the environment.

7. The apparatus of claim 6 wherein the insert is fabricated from quartz or fused silica.

8. The apparatus of claim 3 wherein the housing comprises a generally annular shaped first portion and a second portion having the shape of a toroidal surface.

9. The apparatus of claim 8 wherein the reflector is secured against the an interior surface of the second portion of the housing, and wherein the UV-LEDs are mounted on a printed circuit board (PCB), the PCB is mounted on an interior surface of the first portion to direct the principal optical axes of the UV-LEDs toward the reflective surface of the reflector.

10. The apparatus of claim 9 wherein the interior surface of the second portion of the housing is shaped to conform to the shape of the reflector.

11. The apparatus of claim 8 wherein the first portion and the second portion are coupleable to one another.

12. The apparatus of claim 4 wherein the UV-LEDs are evenly angularly spaced around the chamber.

13. The apparatus of claim 2 wherein:
the fluid dispenser is generally tubular shaped and has a central axis oriented in a direction of a bore of the tubular shape;
the one or more UV radiation emitters comprise a UV-LED having a principal optical axis central to its direction of radiation emission; and
the principal optical axis of the UV-LED is optically oriented toward the fluid dispenser and parallel to the central axis.

14. The apparatus of claim 13 wherein the UV-LED is located or locatable at a location on the central axis.

15. The apparatus of claim 14 comprising a stage operable to move the UV-LED away from the location when the fluid dispenser is in use.

16. The apparatus of claim 13 further comprising a sleeve made of a UV transparent material attached to the exterior perimeter surface of the fluid dispenser.

17. The apparatus of claim 16 wherein the sleeve has an index of refraction in the range of 1.40 to 1.55.

18. The apparatus of claim 2 wherein the fluid dispenser is a nozzle, a spout or a faucet of a fluid-dispensing device.

19. The apparatus of claim 2 wherein the reflective surface comprises a concave surface extending around the exterior perimeter surface of the fluid dispenser.

20. The apparatus of claim 2 wherein the reflective surface comprises a planar, elliptical or parabolic surface extending around the exterior perimeter surface of the fluid dispenser.

21. The apparatus of claim 2 further comprising magnetic means, mechanical means, snap-together means and/or adhesive means for coupling the apparatus to the fluid dispenser or to another object so as to be suitably located adjacent to the fluid dispenser.

22. The apparatus of claim 1 further comprising:
a sensor for detecting a presence of a foreign object in the vicinity of the fluid dispenser; and
control logic configured to turn the UV radiation emitters ON or OFF based on the output from the sensor.

23. The apparatus of claim 2 wherein:
the fluid dispenser is generally tubular shaped and has a central axis oriented in a direction of a bore of the tubular shape; and
the reflective surface extends around an exterior perimeter surface of the fluid dispenser in a direction generally orthogonal to the central axis.

24. The apparatus of claim 23 wherein the reflective surface comprises a cross-sectional shape, in a cross-section orthogonal to the central axis, which is complementary to the external perimeter surface of the fluid dispenser.

25. The apparatus of claim 24 wherein a cross-sectional shape, in a cross-section orthogonal to the central axis, of the external perimeter surface of the fluid dispenser is circular.

26. The apparatus of claim 24 wherein a cross-sectional shape, in a cross-section orthogonal to the central axis, of the external perimeter surface of the fluid dispenser is non-circular (e.g. polygonal).

27. A fluid-dispensing apparatus comprising:
a nozzle operative to dispense a fluid;
a reflector having a reflective surface extending around an exterior perimeter (e.g. circumferential) surface of the nozzle and configured to direct ultraviolet radiation toward the exterior perimeter surface; and
one or more UV radiation emitters optically oriented to emit the ultraviolet radiation toward the reflective surface.

28. A method of retrofitting the apparatus of claim 27 to a fluid-dispensing apparatus operable to dispense fluids from the fluid dispenser, the method comprising coupling the reflector to the fluid dispenser or to another object so as to be suitably located adjacent to the fluid dispenser.
